(19) United States
(12) Patent Application Publication
(10) Pub. No.: US 2005/0261783 A1
(43) Pub. Date: Nov. 24, 2005

(54) Multi-axial fitting with shock absorption for prosthetic foot

(75) Inventors: Bret J. Geilman, North Ogden, UT (US); Eric W. Rubie, Salt Lake City, UT (US)

Correspondence Address: FAEGRE & BENSON LLP;PATENT DOCKETING, 2200 WELLS FARGO CENTER, MINNEAPOLIS, MN, 55402, US

(73) Assignee: Otto Bock HealthCare LP, Minneapolis, MN (US)

(21) Appl. No.: 11/132,567

(22) Filed: May 19, 2005

Related U.S. Application Data
(60) Provisional application No. 60/572,322, filed on May 19, 2004.

(51) Int. Cl. A61F 2/66; A61F 2/62

(52) U.S. Cl. 623/52; 623/38; 267/140.2

(57) ABSTRACT

A fitting for a prosthetic foot permits multi-axial movement of the prosthetic foot with respect to a lower limb prosthesis and provides shock absorption. The multi-axial fitting has a mount, a base, and a shock absorbing resilient member disposed between the mount and the base. The resilient member is torqueable and compressible under force, providing shock absorption, rotation, medial lateral movement and dorsiflexion of the prosthetic foot with respect to the lower limb prosthesis.

### CROSS REFERENCE TO RELATED APPLICATION

The present application claims the benefit of U.S. Provisional Application Ser. No. 60/572,322 entitled “Multi-Axial Fitting with Shock Absorption for Prosthetic Foot,” filed May 19, 2004, which is herein incorporated by reference.

### TECHNICAL FIELD

This invention relates generally to orthopedic prosthetics, and more particularly to fittings for prosthetic feet.

### BACKGROUND

One feature of the human ankle is its ability to permit movement in multiple axes. For example, the ankle permits dorsiflexion/plantarflexion, or the flexing of the foot up and down, medial/lateral movement, or the rolling of the ankle to the sides, and torsion, or rotating the foot with respect to the lower leg. These movements are especially important when traveling over uneven surfaces or engaging in generally athletic activities. In particular, they permit the lower leg to remain relatively stable even as the foot moves over uneven terrain.

In lower leg amputees, a lower limb prosthesis is fastened over the lower leg residuum and coupled to a prosthetic foot to form a prosthetic lower extremity. The prosthetic lower extremity allows an amputee self-propelled ambulation. Prosthetic “ankle” fittings are generally used to couple the prosthetic foot to the lower limb prosthesis. Thus, it is important that prosthetic fittings be capable of imitating the human ankle as much as possible to provide the most natural and stable gait or stride, particularly when traveling over uneven surfaces and engaging in athletic activities.

### SUMMARY

In one embodiment, the present invention is a multi-axial fitting for use with a lower limb prosthesis. The fitting includes a base and a mount, each adapted for coupling to a lower limb prosthesis, an intermediate member fixed to the mount and pivotally and rotatably coupled to the base, and a resilient member disposed between the mount and the base.

In one embodiment, at least a portion of the resilient member is fixed to the base and to the mount while a central portion is compressible and torqueable. The intermediate member includes a bolt having a shaft fixed to the mount and a head movable within a cavity formed under a central portion of the base. A spherical washer may be disposed between the bolt head and the base within the cavity. The bolt head is movable within the cavity relative to the base.

In another embodiment, the intermediate member includes a shaft member and a hinge member. The hinge member is pivotally coupled to the base. The shaft member is fixed to the mount and has a shaft portion slidable within a slot formed in the hinge member. A resilient member may be disposed between the mount and the hinge member, the hinge member and the base, or both. The hinge member is pivotal relative to the base about an axis extending in a lateral direction to permit the base to move relative to the mount in plantarflexion and dorsiflexion.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a side view of a prosthetic lower extremity.

FIG. 2 is a cross-sectional view of a first embodiment of a multi-axial shock-absorbing fitting according to the present invention.

FIG. 3 is a side cut-away view detailing the mount shown in FIG. 2.

FIG. 4 is a perspective view detailing the base shown in FIG. 2.

FIG. 5 is a side view of the multi-axial fitting shown in FIG. 2.

FIG. 6 is an exploded cross-sectional view of a second embodiment of a multi-axial shock-absorbing fitting according to the present invention.

FIG. 7 is a top view detailing the mount shown in FIG. 6.

FIG. 8 is a top view detailing the first bumper shown in FIG. 6.

FIG. 9 is a perspective view detailing of the rocker shown in FIG. 6.

FIG. 10 is a perspective view detailing the base shown in FIG. 6.

FIG. 11 is a perspective view detailing the rocker installed on the base shown in FIG. 6.

FIG. 12 is a perspective view of a third embodiment of a multi-axial shock-absorbing fitting according to the present invention.

FIG. 13 is a perspective view of a fourth embodiment of a multi-axial shock-absorbing fitting according to the present invention.

FIG. 14 is a perspective view of a fifth embodiment of a multi-axial shock-absorbing fitting according to the present invention.

FIG. 15 is a perspective view of a sixth embodiment of a multi-axial shock absorbing fitting according to the present invention.

FIG. 16 is an exploded view of the fitting of FIG. 15.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

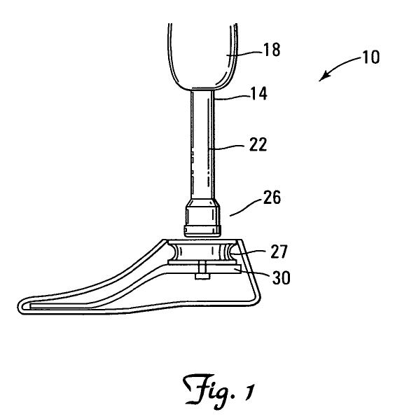

FIG. 1 illustrates a prosthetic lower extremity 10 of the type worn by lower leg amputees. A lower limb prosthesis 14 has a socket 18 securely fastened over a portion of the wearer's lower leg residuum. A pylon 22 is coupled to the socket 18 and extends longitudinally, terminating in an intermediate fitting 26. The intermediate fitting 26 couples the pylon 22 to a multi-axial fitting 27, which is coupled to a mounting portion 31 of a prosthetic foot 30. In combination, the lower limb prosthesis 14, the intermediate fitting 26, the multi-axial fitting 27 and prosthetic foot 30 provide the means for permitting self-propelled ambulation in a lower leg amputee. In one embodiment, the multi-axial fitting 27 is integrated into the prosthetic foot 30. In another embodiment, the multi-axial fitting 27 is integrated into the prosthetic fitting 26. In yet another embodiment, the multi-axial fitting 27 is removably coupled between the prosthetic fitting 26 and the prosthetic foot 30.

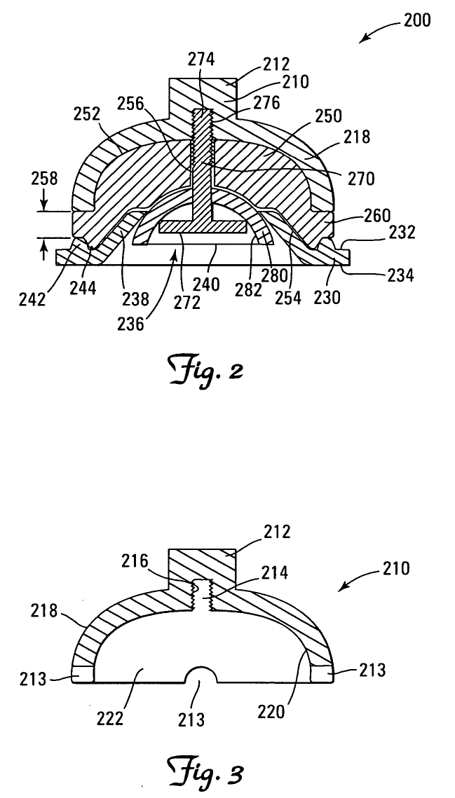

FIG. 2 illustrates a first embodiment of a multi-axial fitting 200 according to the present invention. The multi-axial fitting 200 has a mount 210, a base 230 and a resilient member 250 interposed between the mount 210 and the base 230. The mount 210 has a first coupling means 212 for coupling the fitting 200 to a prosthetic fitting 26 (See FIG. 1). FIG. 3 shows the mount 210 in detail. The first coupling means 212 may be a standard-type pyramid, as shown in FIG. 2, a bolt, a wedge, an interlock, a magnet or other type of fastener. Mount 210 has a mount aperture 214 provided with mounting threads 216. Extending from the first coupling means 212 is a downwardly curving flange 218. An underside 220 of the flange 218 defines a generally semi-hemispherical housing 222.

Returning to FIG. 2, the resilient member 250 is a generally annular member having an upper surface 252, a lower surface 254 and a central bore 256 extending from the lower surface 254 to the upper surface 252. The resilient member 250 is sized and shaped so that the upper surface 252 partially resides within the housing 222. The lower surface 254 of the resilient member 250 rests on an upper surface 232 of the base 230. The resilient member 250 is sized and shaped so that the flange 218 is spaced apart from the upper surface 232 of the base 230, defining a gap 258 between the flange 218 and the upper surface 232 of the base 230. The resilient member 250 is provided with a ring-like protrusion 260 adapted to protrude into the gap 258.

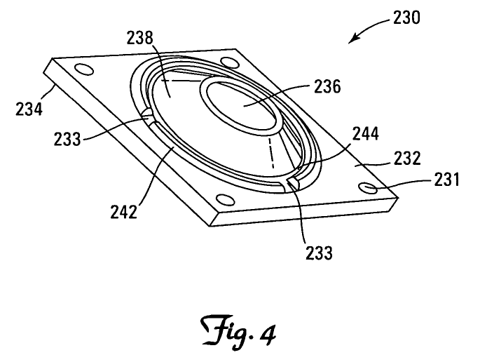

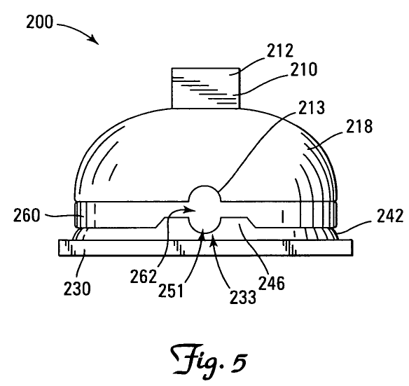

As shown in FIGS. 2 and 4, the base 230 is a generally plate-like member adapted for coupling the multi-axial fitting 200 to a mounting portion 31 of a prosthetic foot 30. The base 230 has a generally flat lower surface 234 adapted as necessary to fit the configuration of the mounting portion 31. For example, the base 230 may be rectangular, as shown, circular or any other shape. The base 230 includes through-holes 231 to facilitate fastening the fitting 200 to the prosthetic foot 30 via fasteners such as screws, bolts or other suitable means. In other embodiments, the base 230 is bonded, adhered or otherwise permanently fastened to the prosthetic foot 30, or is integral with the prosthetic foot 30. Optionally, lower surface 234 includes structure for coupling the base 230 to the prosthetic foot 30, for example a standard type “female” type fitting.

The base 230 includes a centrally located base aperture 236. An annular region of the base 230 surrounding the base aperture 236 is configured at an upward angle, forming a rim or lip 238 on an upper surface 232 of the base 230. The rim 238 defines a cavity 240 between the rim 238 and the lower surface 234 of the base 230. A circular ridge 242 is provided on the base upper surface 232 and is axially aligned with but spaced apart from the rim 238, defining a trough 244. A portion of the lower surface 254 of the resilient member 250 resides within the trough 244, which assists in preventing the resilient member 250 from dislocating from the base 230. The ridge 242, along with the flange 218, defines the aforementioned gap 258 between the mount 210 and the base 230.

The mount 210 includes four mount notches 213 and the base 230 includes four base notches 233 in the flange 218 and upper surface 232, respectively. The resilient member 250 has four corresponding interlocking members 251 sized to be received in the aligned notches 213 and 233. In combination, the notches 213, 233 and interlocking members 251 prevent rotation of the mount 210 with respect to the resilient member 250 and rotation of the resilient member 250 with respect to the base 230.

The mount 210, the resilient member 250, and the base 230 are coupled to one another via a bolt 270 and a spherical washer 280. The spherical washer 280 has a curved inner profile 282. A head portion 272 of the bolt 270 resides within the spherical washer 280. The washer 280 is positioned within the cavity 240. A shaft portion 274 of the bolt 270 is provided with threads 276 and extends upward through the washer 280 and into the mount aperture through the base aperture 236 and the resilient member bore 256. The bolt 270 is secured to the mount 210 via the threads 276. When a downward force is applied to the mount 210, for example, when the wearer transfers weight to the prosthetic foot 30, the resilient member 250 compresses, absorbing some of the force. As the resilient member 250 compresses, the mount 210 migrates toward the base 230, as does the bolt 270. In particular, the bolt 270 moves within the cavity 240 with respect to the base 230. The foregoing arrangement advantageously provides a secure mechanical coupling between the mount 210 and the base 230 while yet permitting movement of the mount 210 with respect to the base 230 when the resilient member 250 compresses and absorbs energy.

When a lateral force is applied to the mount 210, for example, when the wearer walks over uneven terrain, or engages in athletic activities, the resilient member 250 undergoes greater compression in some regions than in others as the force is absorbed. That is, the resilient member 250 compresses on generally the left side or the right side (from the wearer's point of view), providing medial/lateral rollover of the lower limb prosthesis 14 with respect to the prosthetic foot 30, or compresses on generally the front or the rear side (from the wearer's point of view), providing dorsiflexion/plantarflexion of the prosthetic foot 30 with respect to the lower limb prosthesis 14. The bolt 270 correspondingly tilts in response to the uneven compression of the resilient member 250. The foregoing are merely by way of example; the resilient member 250 is compressible all about its circumference, advantageously providing 360 degrees of movement. The spherical washer 280 curved inner profile 282 permits tilting of the bolt head 272 all about its circumference. The amount of compression in a particular region of the resilient member 250 is dependent upon the characteristics of the material and the dimensions of the resilient member 250. Generally, a thicker region of the resilient member 250 is capable of compressing more than a thinner region.

According to one embodiment, as shown in FIG. 4, the ridge 242 has one or more raised areas 246. The ring 260 of the resilient member 250 is provided with a recess 262 adapted to accommodate the raised area 246. The ring 260 is thinnest area of the resilient member 250 between the mount 210 and the base 230. Thus, the maximum amount of compression of the resilient member 250 is limited by the dimensions of the ring 260. The ring 260 is thinner at the recess 262 and thus less subject to compression, reducing movement of the lower limb prosthesis with respect to the prosthetic foot in that area. By way of example, if the raised area 246 was in the “front” of the fitting (from the wearer's point of view), dorsiflexion/plantarflexion is reduced. This feature advantageously permits the prosthetic foot 30 to remain in a stable position when the user travels over uneven terrain or engages in athletic movement. According to other embodiments, the recess 262 and corresponding raised area 246 are positioned at varying locations around the fitting 200 and have varying dimensions.

When torsional force is applied to the mount 210, for example when the wearer exerts rotational force on the lower limb prosthesis 14 while the prosthetic foot 30 is stationary, the torsional force is transferred generally evenly from the first coupling means 212 to the flange 218. The flange 218 in turn transfers torsional force to the upper surface 252 and to the interlocking member 251 of the resilient member 250. In other words, the resilient member lower surface 254 and the interlocking member 251 remain immovably fixed to the base 230, while the resilient member 250 twists. The interlocking member 251 in the resilient member 250 deforms, absorbing the torsional force. As the force is released, the interlocking member 251 returns to its original shape. The bolt 270 and spherical washer 280 rotate along with the resilient member 250 with respect to the base 230 within the cavity 240. This feature advantageously provides rotational movement of the lower limb prosthesis 14 with respect to the prosthetic foot 30.

The mount 210 and base 230 are constructed of rigid materials able to accommodate the wearer's weight and activity level. Examples include titanium, steel, stainless steel, aluminum, composite, cast metal or molded composite or any other like material.

The resilient member 250 is constructed of urethane, elastomer, rubber, silicone or other durable foam-type material capable of resilient compression and torsion. In particular, the resilient members 250 is adapted to compress when subject to force, for example, when the wearer transfers weight to the prosthetic lower extremity.

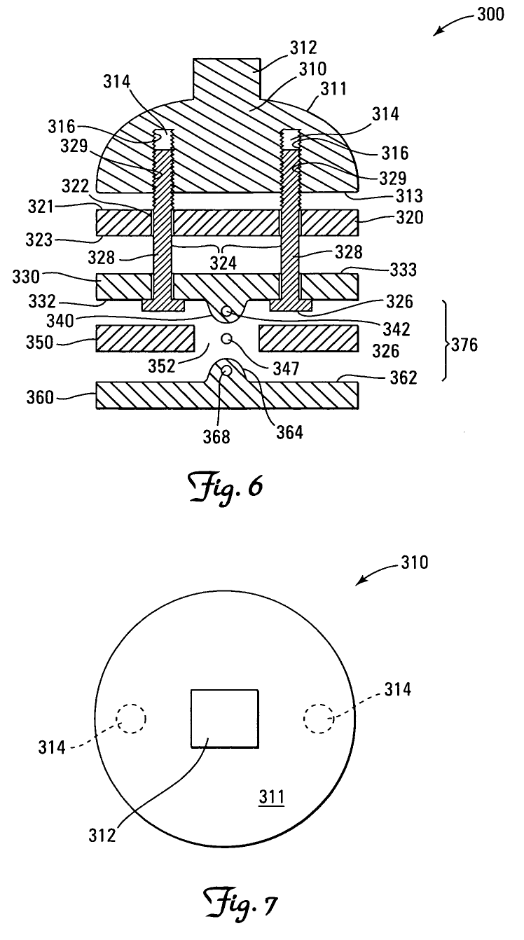

FIGS. 6-11 illustrate a second embodiment of a multi-axial fitting 300 according to the present invention. The fitting 300 is multi-layered and includes a mount 310, a first bumper 320, a rocker 330, a second bumper 350 and a base 360, all stacked one atop the other.

The mount 310 as shown in FIGS. 6 and 7 is generally hemispherical with a generally convex upper surface 311 on which is formed or provided a first coupling means 312 adapted to couple the mount 310 to a prosthetic fitting 26. First coupling means 312 may be a pyramid, as shown in FIG. 6, or any other type of fastener as previously discussed. The mount 310 has a pair of mount apertures 314 formed in a lower surface 313, each provided with inner threads 316.

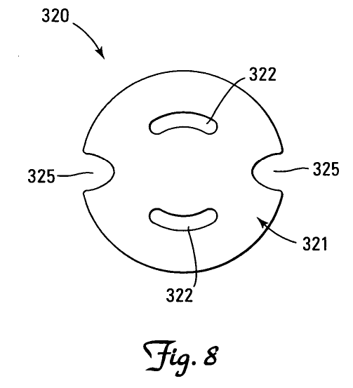

FIG. 8 illustrates first bumper 320, a generally circular disc having an upper surface 321 adjacent the mount 310 and a lower surface 323 adjacent the rocker 330. A pair of kidney-shaped through-holes 322 extend through the first bumper 320 and are aligned with the mount apertures 314. The first bumper 320 is further provided with a pair of inwardly protruding notches 325.

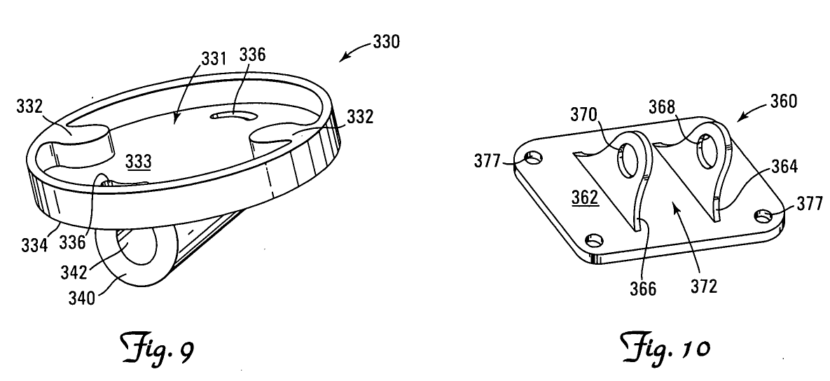

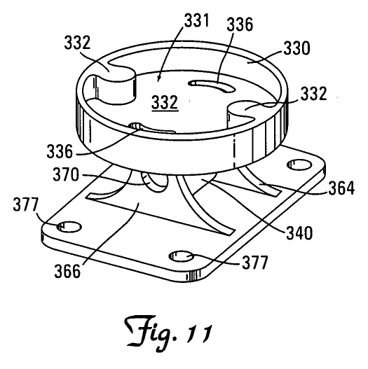

The rocker 330, as shown in detail in FIG. 9, is a generally circular member defining a cup area 331 adapted for receiving the first bumper 320. A pair of inwardly protruding tongues 332 are received by the notches 325 of the first bumper 320 to interlock the first bumper 320 to the rocker 330. Two spaced apart kidney shaped apertures 336 extend through rocker 330 from a lower surface 332 to an upper surface 334. The apertures 336 are generally mirror images of one another and are aligned with the apertures 322 of the first bumper 320.

The rocker 330 is coupled to the mount 310 and first bumper 320 by a fastener, for example, shoulder bolt 324. Shoulder bolts 324 each have a head portion 326 and a shank portion 328 provided with threads 329 complementary to inner mount inner threads 316. The head portion 326 resides at the lower surface 334 of the rocker 330 while the shank portion 328 extends through the rocker apertures 336, through the first bumper apertures 322 and into the mount apertures 314.

The shoulder bolt 324 is coupled to the mount 310 via the threads 316 and 329. The shoulder bolt head 326 is slidable along the lower surface 334 of the rocker 330 the length of the rocker apertures 336 so that the lower surface 313 of the mount 310 slides over the upper surface 321 of the first bumper 320. This feature advantageously provides a rotatable coupling between the mount 310 and the rocker 330. The degree of rotation permitted depends on the length of the rocker apertures 336 and the first bumper apertures 322, and may be adapted to accommodate the needs of various users.

The lower surface 334 of the rocker 330 is provided with a hinge member 340. Hinge member 340 is a generally curved protrusion having a orifice or hole 342 therethrough. As shown in FIGS. 10 and 11, an upper surface 362 of the base 360 is provided with two hinge receiving members 364, 366 with orifices or holes 368, 370 therethrough. The hinge receiving members 364, 366 are spaced apart and define a gap 372 therebetween and are positioned so that the orifices 368, 370 are aligned. The gap 372 is adapted to receive the hinge member 340 so that the hinge member orifice 342 is aligned with both hinge receiving member orifices 368, 370. A pin 347 resides within the aligned orifices 342, 368 and 370 and couples the rocker 330 to the base 360. The combination of the hinge member 340 and hinge receiving members 364, 366 form a hinge 376 which pivotably couples the rocker 330, and thus the mount 210, to the base 360.

The pivot or rocking feature advantageously provides, for example, medial/lateral motion or dorsiflexion/plantarflexion, of the mount 310, and thus the prosthetic leg 14, with respect to the base 360, and thus the prosthetic foot 30. The base 360 is further provided with means 377 of coupling the fitting 300 to a prosthetic foot 300. As shown in FIG. 11, means 377 are through-holes, or another fastener such as screw, bolts or other suitable means. In other embodiments, the base 360 is bonded, adhered or otherwise permanently fastened to the prosthetic foot 30, or is integral with the prosthetic foot 300.

The second bumper 350 is a ring-like member having a ring opening 352 located in between the rocker 330 and the base 360. The hinge 376 is located in the ring opening 352. The second bumper 350 is made of a resilient material adapted to cushion rocking forces between the rocker 330 and the base 360. The second bumper 350 advantageously provides shock absorption and a smooth rocking motion as the rocker 340 pivots about the hinge 376.

The mount 310, rocker 330 and base 360 are constructed of rigid materials able to accommodate the wearer's weight and activity level. Examples include titanium, steel, stainless steel, aluminum, composite, cast metal or molded composite or any other like material.

The first and second bumpers 320 and 350 are constructed of urethane, elastomer, rubber, silicone or other durable foam-type material capable of resilient compression and torsion. In particular, the first and second bumpers 320 and 350 are adapted to compress when subject to force, for example, when the wearer transfers weight to the prosthetic lower extremity.

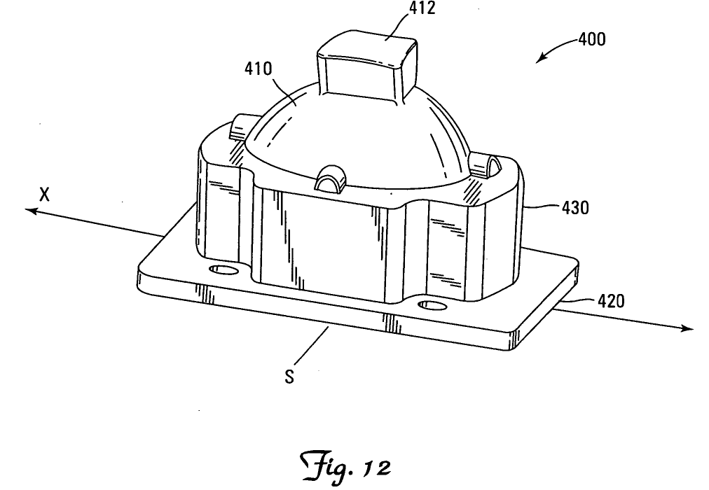

FIG. 12 shows a perspective view of a multi-axial fitting 400 according to a third embodiment of the present invention. The fitting 400 includes a mount 410, including a coupling means 412, a base 420 and a resilient member 430 interposed between the mount 410 and the base 420. The resilient member 430 has a generally oblong shape defining a long axis “X” and a short axis “S”. In particular, a fitting 400 according to the present embodiment advantageously provides increased resistance in the longer “X” axis and reduced resistance in the shorter “S” axis. For example, a fitting 400 according to the present embodiment reduces dorsiflexion/plantarflexion in the front and rear regions while providing increased medial/lateral motion. Preferably, the ratio of the resistance of the long “X” axis with respect to the short “S” axis is sized to an individual user's requirements.

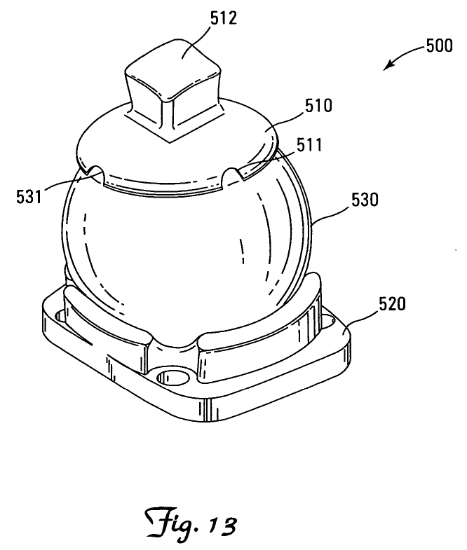

FIG. 13 shows a perspective view of a multi-axial fitting 500 according to a fourth embodiment of the present invention. Fitting 500 has a mount 510, a base 520 and a resilient member 530 interposed between the mount 510 and the base 520. The resilient member 530 is shown having a generally spheroid shape. It is contemplated that the resilient member 530 also have a disc or rectangular shape. The resilient member 530 is further provided with a plurality of tongues 531 protruding outwardly adjacent the mount 510. The mount 510 is likewise provided with corresponding inwardly protruding notches 511 sized to receive the tongues 531. The tongues 531 and notches 511 interlock to prevent rotation of the mount 510 with respect to the resilient member 530.

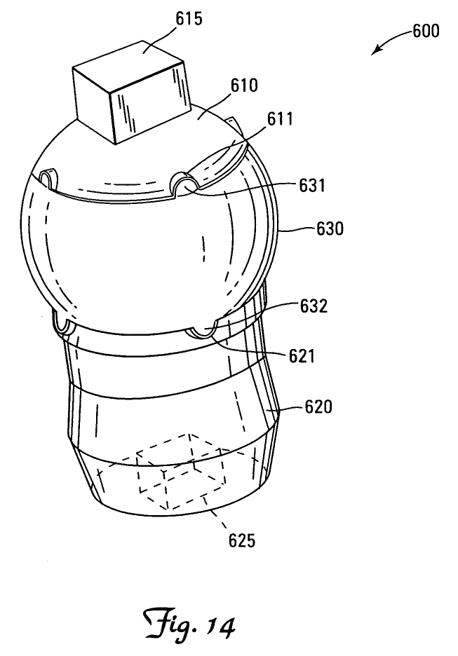

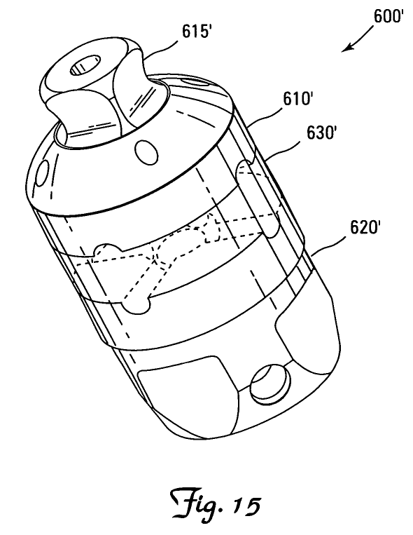

FIG. 14 shows a perspective view of a multi-axial fitting 600 according to a fifth embodiment of the present invention. The fitting 600 includes a mount 610, a base 620 and a resilient member 630 interposed between the base 620 and the mount 610. The mount 610 includes a first coupling means 615 for coupling the fitting 600 to the mounting portion of a prosthetic lower extremity, for example a socket or a pylon of a lower limb prosthesis, or a prosthetic fitting. The first coupling means 615 includes but is not limited to a standard-type pyramid, a bolt, a hook, a pin or other type of fastener.

The resilient member 630 is further provided with a plurality of tongues 631, 632 protruding outwardly adjacent the mount 610 and base 620, respectively. The mount 610 is likewise provided with corresponding inwardly protruding notches 611 sized to receive the tongues 631. The tongues 631 and notches 611 interlock to prevent rotation of the mount 610 with respect to the resilient member 630. Notches 621 protrude inwardly from the base 620 adjacent the resilient member 630. The notches 621 and tongues 632 likewise interlock to prevent rotation of the resilient member 630 with respect to the base 620.

The base 620 includes a second coupling means 625 complementary to the first coupling means 615 for coupling the fitting 600 to a prosthetic lower extremity, for example a socket or a pylon of a lower limb prosthesis, prosthetic fitting or prosthetic foot. The second coupling means 625 is thus configured generally similar to the mounting portion of the prosthetic lower extremity coupled to the mount 610. This feature advantageously provides a universal type fitting such that the fitting is mountable in a variety of configurations. For example, a fitting according to the present embodiment is mountable to a prosthetic knee or socket as well as to a prosthetic ankle or prosthetic foot.

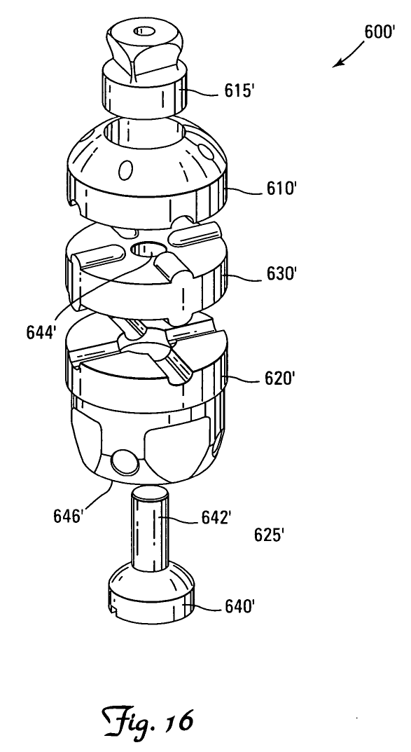

FIGS. 15 and 16 show a perspective view and an exploded view of a multi-axial fitting 600′ according to a sixth embodiment of the present invention. Fitting 600′ is generally similar to the fitting 600 shown in FIG. 14 and like parts are given like numbering. However, as is shown in FIGS. 5 and 16, the resilient member 630′ is shaped like a disc rather than the bulging or spherical shape of the resilient member 630 of the embodiment shown generally in FIG. 14. Furthermore, as is shown in FIGS. 15 and 16, the base 620′ includes a second coupling means 625′ that is somewhat different that the second coupling means 625 of the embodiment shown generally in FIG. 14. Second coupling means 625′ includes an adaptor 640′ and a stem 642′. The stem 642′ extends upwardly through a bore 644′ in the resilient member 630′. The adaptor 640′ is received in a recess (not visible) in a lower surface 646′ of the base 620′ so as to be substantially flush with the lower surface 646′.

Although the present invention has been described with reference to exemplary embodiments, persons skilled in the art will recognize that changes may be made in form and detail without departing from the spirit and scope of the invention. In addition, the various embodiments described include numerous components which may be provided in various combinations to achieve similar functionality. All such combinations are within the scope of the present invention. Also, various of the components may be eliminated from one or more embodiments to achieve the same function, as described above.

1. A multi-axial fitting for use with a lower limb prosthesis, the fitting comprising:
a base adapted for coupling to a lower limb prosthesis;
a mount adapted for coupling to a lower limb prosthesis;
an intermediate member fixed to the mount and pivotally and rotatably coupled to the base; and
a resilient member disposed between the mount and the base.

2. The multi-axial fitting of claim 1 wherein the mount includes a pyramid for coupling the fitting to a lower limb prosthesis.

3. The multi-axial fitting of claim 1 wherein the base includes a pyramid for coupling the fitting to a lower limb prosthesis.

4. The multi-axial fitting of claim 1 wherein a first region of the resilient member is fixed to the mount, a second region of the resilient member is fixed to the base and a central region of the resilient member is compressible and torqueable.

5. The multi-axial fitting of claim 4 wherein:
the base has an aperture extending through a central region thereof and an annular region surrounding the aperture is angled upwardly, such that a lower surface of the base at the annular region defines a cavity; and
the intermediate member comprises a bolt coupling the mount and to the base, wherein the bolt has a circular head positioned in the recess and a shaft extending through the resilient member and is fixedly engaged to the mount.

6. The multi-axial fitting of claim 5 wherein the head of the bolt is movable within the recess.

7. The multi-axial fitting of claim 5 wherein the bolt is movable relative to the resilient member.

8. The multi-axial fitting of claim 5 further comprising a spherical washer interposed between the bolt head and the base aperture.

9. The multi-axial fitting of claim 5 wherein the mount has a substantially hemispherical shape defining a recess having concave inner side walls and the resilient member has an upper portion shaped to be received in the recess.

10. The multi-axial fitting of claim 5 wherein at least a portion of the resilient member is longer along an axis extending in a fore and aft direction than in an axis extending in a lateral direction.

11. The multi-axial fitting of claim 5 wherein at least a portion of the resilient member is substantially spherical.

12. The multi-axial fitting of claim 1 wherein the intermediate member comprises a shaft member coupled to the mount and a hinge member pivotally coupled to the base, wherein the shaft member is slidable within a slot formed in the hinge member.

13. The multi-axial fitting of claim 12 wherein the slot is kidney shaped.

14. The multi-axial fitting of claim 12 wherein the hinge member is pivotable relative to the base about an axis extending in a lateral direction to permit dorsiflexion and plantarflexion of the base relative to the mount.

15. The multi-axial fitting of claim 12 wherein the resilient member is disposed between the mount and the hinge member.

16. The multi-axial fitting of claim 12 wherein the resilient member is disposed between the base and the hinge member.

17. The multi-axial fitting of claim 16 wherein a portion of the shaft is slidable over the resilient member.

18. A multi-axial fitting for use with a lower limb prosthesis, the fitting comprising:
a base adapted for coupling to a lower limb prosthesis, wherein the base has an aperture extending through a central region thereof and an annular region surrounding the aperture is angled upwardly, such that a lower surface of the base at the annular region defines a cavity;
a mount adapted for coupling to a lower limb prosthesis;
an intermediate member fixed to the mount and pivotally and rotatably coupled to the base, wherein the intermediate member comprises a bolt coupling the mount and to the base, wherein the bolt has a circular head positioned in the recess and a shaft extending through the resilient member and is fixedly engaged to the mount; and
a resilient member disposed between the mount and the base.

19. The multi-axial fitting of claim 18 further comprising a spherical washer interposed between the bolt head and the base aperture.

20. The multi-axial fitting of claim 18 wherein the mount has a substantially hemispherical shape defining a recess having concave inner side walls and the resilient member has an upper portion shaped to be received in the recess.

21. A multi-axial fitting for use with a lower limb prosthesis, the fitting comprising:
a base adapted for coupling to a lower limb prosthesis;
a hinge member pivotally coupled to the base;
a mount rotably coupled to the hinge member with a shaft member, the shaft member having a shaft portion slidable within a slot extending through the hinge member; and
a first resilient member disposed between the base and the hinge member and a second resilient member disposed between the mount and the hinge member.

22. The multi-axial fitting of claim 21 wherein the slot is kidney shaped.

23. The multi-axial fitting of claim 21 wherein the shaft member comprises a pair of opposing shaft portions each slidable within a corresponding slot extending through the hinge member.

24. The multi-axial fitting of claim 21 wherein the hinge member is pivotable relative to the base about an axis extending in a lateral direction to permit dorsiflexion and plantarflexion of the base relative to the mount.
